(19) United States
(12) Plant Patent Application Publication
(10) Pub. No.: US 2008/0320629 P1
(43) Pub. Date: Dec. 25, 2008

(54) Verbena plant named 'Scarlet'

(75) Inventor: Jurgen Weiss, Goch (DE)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/820,920

(22) Filed: Jun. 21, 2007

(51) Int. Cl. A01H 5/00

(52) U.S. Cl. PLT/308

(57) ABSTRACT

A new and distinct variety of Verbena plant particularly distinguished by its scarlet flower, early flowering, and a habit that is compact and spreading.

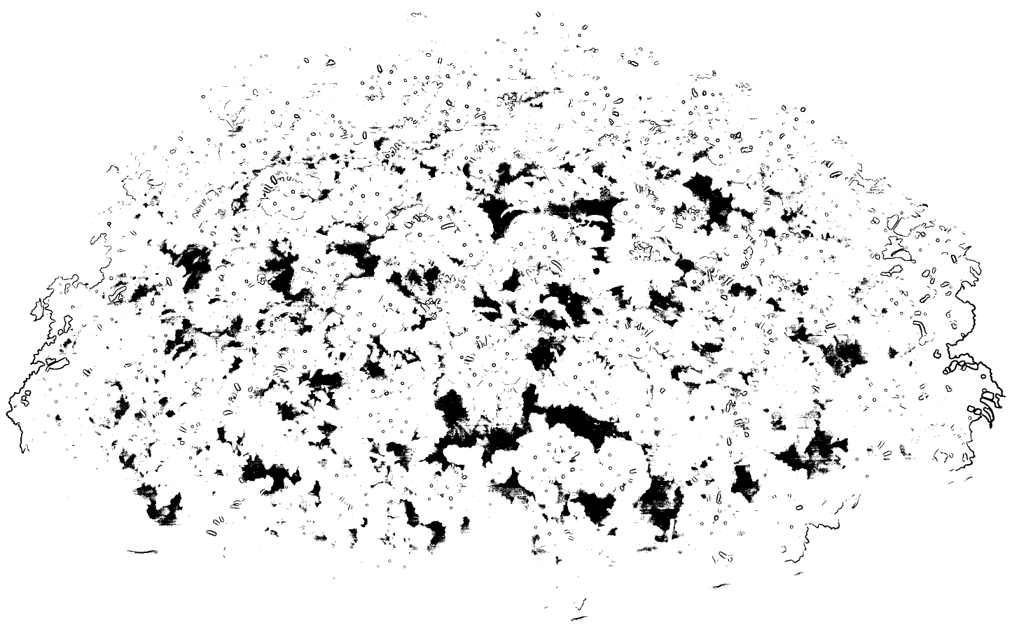

### LATIN NAME OF THE GENUS AND SPECIES OF THE PLANT CLAIMED

Verbena×hybrida

### VARIETAL DENOMINATION

‘Scarlet’

### BACKGROUND OF THE NEW PLANT

The present invention comprises a new distinct cultivar of Verbena, botanically known as Verbena×hybrida.

The new cultivar is originated from a found seedling in Kleve, Germany. As a result of this finding the present cultivar was created in 2002 in Enkhuizen, Netherlands and has been repeatedly asexually reproduced by cuttings in Enkhuizen, Netherlands and Sarrians, France over a two year period. The new variety is stable and reproduces true to type in successive generations of asexual reproduction.

This new Verbena plant is an annual in most climatical zones in the US, only in zones 8, 9 and 10 it is a perennial plant.

### DESCRIPTION OF THE DRAWING

This new Verbena plant is illustrated by the accompanying photographic drawing which shows blooms, buds and foliage of the plant in full color, the color shown being as true as can be reasonably obtained by conventional photographic procedures.

### DESCRIPTION OF THE NEW CULTIVAR

The following detailed descriptions set forth the distinctive characteristics of this new Verbena. The data that define these characteristics were collected from asexual reproductions carried out in Enkhuizen, Netherlands. The plant history was taken on 14 week old plants, blossomed under natural light in a greenhouse and grown in a 10.5 cm container.

Color references are primarily to the R.H.S. Colour Chart of The Royal Horticultural Society of London.

TABLE 1
DIFFERENCES BETWEEN THE NEW CULTIVAR
‘SCARLET’ AND A SIMILAR CULTIVAR

| | ‘Scarlet’ | ‘Scarlena’ |
|---|---|---|
| Flower size | 14-15 mm | 17-19 mm |
| Leaf size | Medium | Large |

The commercial name of the most resembling variety is ‘Tukana Scarlet.’ This variety has the variety name ‘Scarlena’ and is patented as U.S. Plant Pat. No. 12,578.
The plant:
   Classification—Botanical.—Verbena×hybrida.
   Parentage.—Unknown, found seedling.
   Growth habit.—Upright, decumbent.
   Plant height.—10-12 cm.
   Spreading area of plant.—20-40 cm.
   Branching character.—Freely branching and lateral branching at every node.
   Number of branches per plant.—24-34.
   Blooming period.—From March until November.
The stem:
   Diameter.—2 mm.
   Length.—10-20 cm.
   Shape.—Tetragonal.
   Anthocyanin pigmentation.—Absent.
   Color of the stem.—146A.
   Length of internode.—15-30 mm.
   Texture.—Smooth.
   Pubescence.—Pubescent.
   Length lateral branches.—18-28 cm.
The foliage:
   Phyllotaxis.—Opposite.
   Shape of blade.—Broadly ovate.
   Texture.—Upper side: Smooth — Lower side: Smooth.
   Venation.—Pinnate.
   Leaf margin.—Dentate.
   Leaf base.—Obtuse.
   Leaf apex.—Acute.
   Length.—20-34 mm.
   Width.—18-20 mm.
   Depth of incision.—2-3mm.
   Number of incisions.—8-16 per leaf.
   Color.—Upper side: 137B — Lower side: 141B.
   Pubescence.—Slightly pubescent.
   Length of petiole.—3 mm.
   Petiole surface structure.—Slightly pubescent.
   Petiole diameter.—1 mm.
   Petiole coloration.—138B, both surfaces.
The bud:
   Peduncle length.—25-35 mm.
   Peduncle diameter.—2.2 mm.
   Peduncle color.—137B.
   Bud size.—Diameter: 2 mm — Length: 4-6 mm.
   Bud shape.—Elongated and ovate.
   Bud color.—47B.
   Sepals.—Color (upper side): 137B — Color (lower side): 137B — Form: Upright — Number: 5, fused — Length: 6-8 mm — Width: 2 mm — Shape: Elongated — Apex: Emarginate — Base: Fused — Margin: Entire.
The flower:
   Flower diameter.—14-15 mm.
   Flower height.—14-22 mm.
   Flower tube length.—12-20 mm.
   Flower throat diameter.—2 mm.
   Flower throat color.—156C.
   Inflorescence.—Corymb.
   Flower-form.—Single, salverform; sessile on terminal corymbs.
   Petal color.—Upper side: 44A — Lower side: 44B.
   Overlapping of petals.—Overlapping.
   No. of petals.—Gamopetalous, 5 lobed.
   Petal apex.—Obcordate.
   Petal base.—Fused.
   Petal margin.—Entire.
   Petal surface texture.—Smooth.
   Size of the petal.—Length: 5-7 mm — Width: 4-6 mm.
   Inflorescence.—Length: 20-30 mm — Diameter: 24-34 mm.
   Calyx length.—7-9 mm.
   Calyx diameter.—3 mm.
   Anthocyanin pigmentation of calyx limb.—Absent.
   Color of the calyx.—137C.
   No. of flowers per inflorescence.—20-35.
   Fragrance.—No fragrance.
   Bloom time of one inflorescence.—New florets continue to open in one corymb over a period of 15 days.
   Lastingness of one flower.—2-4 days.
Reproductive organs:
   Androecium.—Stamens quantity: 4 Anther shape: Ovoid Anther length: 1 mm Anther color: 144B Pollen amount: No pollen.
   Gynoecium.—Pistils quantity: 1 Pistil length: 1-1.5 cm Stigma shape: Bi-lobed Stigma color: 144C Style length: 1.9 cm Style color: 144C Ovary color: 144C.
The fruit and the seed:
   Seed set.—Little fruit development and seed set has been observed to date.
   Seed shape.—Elongated.
   Length.—3.5 mm.
   Width.—1 mm.
   Color.—166C.
Roots:
   Type of roots.—Fibrous, color 155B. Roots start to grow on ever part of the stem that contacts the soil, so not only at the nodes.
Physiological and ecological characteristics:
   Disease/pest resistance.—Plants of the new Verbena have been noted to be resistant to especially powdery mildew.
   Temperature tolerance.—Plants of the new Verbena have been observed to tolerate temperatures from 2-35 degrees C.

What is claimed is:

1. A new and distinct variety of Verbena plant named ‘Scarlet,’ substantially as herein illustrated and described.
